(19) United States
(12) Patent Application Publication
(10) Pub. No.: US 2024/0070437 A1
(43) Pub. Date: Feb. 29, 2024

(54) METHOD FOR CORRECTING A DETECTED RESULT, DETECTION ARRANGEMENT, PHANTOM, AND APPARATUS FOR GENERATING SYNTHETIC DATA PAIRS

(71) Applicant: PTW-Freiburg Physikalisch-Technische Werkstätten Dr. Pychlau GmbH, Freiburg (DE)

(72) Inventors: Jan Weidner, Freiburg (DE); Daniela POPPINGA, Berlin (DE)

(73) Assignee: PTW-Freiburg Physikalisch-Technische Werkstätten Dr. Pychlau GmbH, Freiburg (DE)

(21) Appl. No.: 18/150,881

(22) Filed: Jan. 6, 2023

(30) Foreign Application Priority Data
Aug. 25, 2022 (DE) 202022104805.3

(51) Int. Cl. G06N 3/045

(52) U.S. Cl. CPC G06N 3/045; G06T 2207/20084

(57) ABSTRACT

A method for correcting a result (5), detected using a detector (2), of a radiation-physics process pertaining to a radiation-source (6) by an artificial neural network (4) is provided. The artificial neural network (4) was initially trained with synthetic data pairs (8), and the synthetic data pairs (8) include a first, in particular synthetic, datum (9) and a second, in particular synthetic, datum (10). The data (9, 10) of a data pair (8) differ by a detector-specific transformation (11) that is uniform for all data pairs (8). A detection arrangement as well as a phantom for use with such a detection arrangement are also provided.

### INCORPORATION BY REFERENCE

The following documents are incorporated herein by reference as if fully set forth: German Patent Application No. 20 2022 104 805.3, filed Aug. 25, 2022.

### TECHNICAL FIELD

The invention relates to a method for correcting a result, detected by means of a detector, of a radiation-physics process pertaining to a radiation-source by an artificial neural network.

The invention further relates to a detection arrangement with a detector and with an arithmetic logic unit with an artificial neural network.

The invention further relates to a detection arrangement and to a use of a detection arrangement.

The invention further relates to a method for measuring radiation with a phantom.

The invention further relates to a phantom with a detection arrangement.

The invention further relates to an apparatus for generating synthetic data pairs.

The subjects of the invention have preferentially been conceived for application in dosimetry in radiotherapy.

### BACKGROUND

In radiotherapy, it is necessary to examine radiation-sources for application on a patient regularly.

The radiation-source is, for instance, a linear accelerator. A radiation-physics process pertaining to a radiation-source may comprise, in particular, the following steps: releasing and accelerating particles, and/or entry and/or passage of particles into and/or through a patient and/or a phantom, and/or detection of a particle beam.

In this connection, a check is made as to whether the dose of radiation stipulated in a treatment plan is actually being adhered to at a particular location on or in the patient. For this purpose, measurements of the dose of radiation take place, as a rule, by means of a detector and using a phantom. A phantom serves to simulate the radiation-absorption behavior of a human being and may comprise, for instance, a water-filled container made of Plexiglas. The phantom may also have been manufactured from a plastic material.

Within the scope of therapy-planning, the stipulated dose of radiation at various locations is calculated, as a rule, on the basis of a beam model. The beam profile, for instance, enters into this model. This profile depends on the type and geometry of the radiation-source and is set, for instance, by means of a multi-leaf collimator. The latter exhibits several pairs of leaves which can be set in a desired position, and the space between them shapes the beam.

In the course of the actual measurement of a beam profile, for instance within the scope of an examination, the detected result is additionally influenced by the geometry of the detector. By virtue of this, the result measured with the detector deviates from the actual beam profile. This effect is particularly pronounced in the penumbral region of the beam profile. By “penumbral region”, that marginal region of the beam profile may be understood in which no sharp edge has been formed when the radiation-source is not punctiform.

The detected result can be corrected for these detector-specific deviations from an ideal. For this purpose, it is conventional to perform reference measurements in order to determine this detector-specific deviation.

### SUMMARY

It is an object of the invention to simplify the correction of the detected result, in order in this way to be able to obtain, more easily and more quickly, more accurate results of measurement in which the detector-specific deviation has already been taken into account.

With a view to achieving the stated object, one or more features of the invention as disclosed herein are provided. Consequently, with a view to achieving the stated object in the case of methods of the type described in the introduction, in accordance with the invention it is proposed, in particular, that the artificial neural network was initially trained with synthetic data pairs, the data pairs comprising a first datum and a second datum, and that the data of a data pair differ by a detector-specific transformation that is uniform for all data pairs.

The correction, performed by the neural network, of the result detected by means of the detector may be employed, for instance, when the radiation-physics process pertaining to the radiation-source is acting on a patient or, for instance, only on a phantom. The accuracy of real measurements or irradiations on the patient, and/or of calibration measurements or test measurements on a phantom, can consequently be improved with the method according to the invention.

The first datum of the data pair may describe, for instance, a synthetic beam profile.

The second datum, on the other hand, may describe, for instance, a synthetic detection result.

Consequently, by “datum” itself here a certain number of data may be understood, or rather, the first datum and/or the second datum may each comprise several data.

By “synthetic”, it may be understood, in particular, that the datum, data pair, profile and/or result in question has/have not been measured but has/have been generated artificially.

An equivalent of the initial training of the artificial neural network is contained in this network, for instance in the form of a certain weighting of its artificial neurons. The artificial neural network may be, for instance, a convolutional neural network.

In this connection, the first datum may be a synthetic beam profile. The synthetic beam profile may describe, for instance, the ideal of a beam profile. Furthermore, the first datum may alternatively be a beam profile measured with a detector with comparatively high accuracy. The second datum may be a synthetic detection result. Furthermore, the second datum may alternatively be a beam profile measured by means of a detector of comparatively low accuracy, or may describe such a profile.

A datum may comprise, for instance, information about an intensity of a radiation at a particular location, for instance at the location of the detector. A datum may also comprise several values of intensities at several locations and may consequently describe a beam profile, for instance between two locations at which the intensity amounts to zero or almost zero.

A synthetic data pair contains at least one synthetic datum. Preferably, both the first and second datum are synthetic. For instance, a second datum can be generated synthetically from an actual result of measurement as first datum. This can be effected by application of a detector-specific transformation, for instance a detector-specific lateral response function, to the first datum. By a “lateral response function” here, in particular a function may be understood that describes the broadening of a penumbral region of a measured beam profile, or transverse dose profile, of high-energy radiation by reason of the finite size of the detector. This broadening of the measured beam profile, or dose profile, can be described mathematically as a convolution of the true, undisturbed or ideal beam profile, or dose profile, with the lateral response function of the detector that was used for measurement. By application of a different detector-specific transformation to the second datum, the first datum can be recovered.

By virtue of the fact that the data of a data pair differ by a detector-specific transformation that is uniform for all data pairs, the artificial neural network can be trained to transform or convert the second datum into the first datum. Consequently the artificial neural network can be used to correct a detected beam profile, which is represented by a second datum, for detector-specific influences. By virtue of the fact that the first datum and/or the second datum may be synthetic data, some or all of the previously necessary reference measurements may be dispensable. Consequently there is a saving of time and resources.

The invention consequently proposes, in particular, to generate a detector-specific deviation—which, for example, can be modeled by a lateral response function—synthetically, and not to measure it under real conditions. This can be effected, for instance, by a detector-specific transformation—that is to say, for instance, a mathematical operation such as a convolution. By this means, it is ensured that the synthetic data pairs that serve as training data for the neural network and that were generated with the method according to the invention or with an apparatus according to the invention also include the influence of the detector that the latter would exert on the real results of measurement. As a result, in this way the trained neural network can correct much more precisely and accurately the result detected by means of the detector.

In an advantageous configuration there may be provision that the second datum is generated at least by means of a convolution of the first datum with a lateral response function.

In practice it is the case, as a rule, that the lateral response function with which an ideal can be convolved in order to obtain the actually detected result is known. In addition to convolution, further mathematical operations may be necessary. Whereas the convolution of the first datum—that is to say, of the ideal—with the lateral response function in order to obtain the second datum is comparatively easy to carry out, the deconvolution of the second datum in order to obtain the first datum is not readily possible. The invention has recognized that a plurality of synthetic second data—for instance, detection results—can be easily generated from a plurality of synthetic first data—for instance, beam profiles. These data can then be utilized for training the artificial neural network which can learn to transform the second datum into the first datum. The artificial neural network trained in this way can subsequently be employed in order to correct actually detected results for detector-specific deviations from an ideal. The invention has recognized that realistic beam profiles, and in particular the penumbral regions thereof, do not necessarily have to be modeled for the purpose of training the artificial neural network. It is sufficient if these profiles can be generalized to real beam profiles or penumbral regions.

In an advantageous configuration there may be provision that the first datum is generated by means of a convolution of a synthetic rough profile with a mapping function.

For instance, the first datum can be generated by means of a convolution of rectangular profiles and/or rectangular functions with Gaussian functions, preferentially of different width and/or standard deviation.

Consequently, it is possible in straightforward manner to specify parameters for a synthetic rough profile and subsequently to convert this profile into a first datum, for instance a synthetic beam profile. These parameters are, for instance, width parameters, height parameters and/or location parameters of the rough profile.

In an advantageous configuration there may be provision that the lateral response function and/or the mapping function are distribution functions. These may, for instance, each be a Gaussian function. The distribution functions may also be the sum and/or a concatenation of several Gaussian functions. For instance, several convolutions with Gaussian functions can be carried out.

It has been shown that the detector-specific deviations of a detected result from an ideal can be modeled as a convolution with a Gaussian function. Consequently this convolution can be utilized to convert first data and second data, for instance to transform synthetic first data such as a synthetic beam profile into synthetic second data such as a synthetic detection result.

Alternatively or additionally, there may be provision that the synthetic rough profile is a piecemeal linear function. The synthetic rough profile may be, for instance, a rectangular profile and/or a rectangular function. The synthetic rough profile may, for instance, be chosen to be narrower than a typical leaf width of a multi-leaf collimator. The synthetic rough profile may furthermore be chosen to be wider than a typical opening cross-section of an aperture. The synthetic rough profile may exhibit, for instance, comparatively few parameters in comparison with the first and second data. The synthetic rough profile may furthermore exhibit supporting points with leaf-width spacing. By means of supporting points with leaf-width spacing, particular values that simulate the position of a leaf of a multi-leaf collimator can be defined or generated for each supporting point.

In an advantageous configuration there may be provision that the synthetic rough profile is randomly generated. In this case, there may alternatively or additionally be provision that the mapping function—alternatively or additionally, the first datum—is randomly generated.

In particular, there may be provision that at least individual (but not necessarily all) parameters of the rough profile and/or of the mapping function and/or of the first datum are randomly selected, preferentially within a respective specified parameter range. Consequently, not all the parameters necessarily have to be chosen randomly in each case. For instance, said parameters may be partially or completely randomly generated, for instance within certain parameter ranges, or partially defined. In this way, said respective parameters may, for instance, be partially or completely randomly generated, for instance within certain parameter ranges, or partially defined (perhaps only within ranges and/or in mutual dependence, and/or only individual parameters but not all).

The convolution of the synthetic rough profile with several Gaussian functions, the parameters of which can be chosen randomly, makes it possible to obtain different forms of the first datum, for instance of the synthetic beam profile and in particular of the penumbral region thereof, randomly. The randomly varying parameters of the Gaussian functions may be, for instance, the standard deviations thereof or a weighting of the various Gaussian functions.

Consequently a plurality of first data and second data can be randomly generated from a synthetic rough profile exhibiting comparatively few parameters.

In an advantageous configuration there may be provision that a plurality of synthetic rough profiles and/or first data and/or second data within a single data set are used for training the artificial neural network. For instance, use may be made of more than 1000, preferably more than 3000, of the respective profiles or data within one data set.

The profiles or data can consequently be combined in one data set in resource-saving manner.

In an advantageous configuration there may be provision that the standard deviations of the distribution functions have been restricted to fixed values and/or ranges of values. In particular, this may relate to the Gaussian functions.

Consequently, values can be chosen that provide realistic results. In practice, it may, for instance, be known that when use is made of a specific detector the deviation of the actually detected result from an ideal can be modeled by means of a convolution of the ideal with a Gaussian function having a certain standard deviation.

In an advantageous configuration there may be provision that the or a synthetic rough profile is furnished with a noise. Alternatively or additionally, there may be provision that the or a first datum and/or the or a second datum is/are furnished with a noise. The noise has preferentially been randomly generated.

Consequently the synthetic data may also exhibit a noise that, due to the instrument, enters into the detected result in an actual detection arrangement. The artificial neural network can consequently be trained to correct this noise too. In addition, the diversity of the stated profiles or data can be enhanced further. In this connection, a noise can, for instance, be introduced already in the case of the synthetic rough profile, or can be applied to the second datum as end result. For instance, a noise of the synthetic rough profile may have been limited to being applied only to values of the profile greater than zero, which may correspond to an at least partially opened aperture of a multi-leaf collimator.

In addition, there may be provision that the noise is greater than to be expected in the case of the detected results to be corrected.

Consequently the robustness of the trained artificial neural network, and consequently of the method according to the invention, can be enhanced.

In an advantageous configuration there may be provision that the or a synthetic rough profile is recalculated locally at changed resolution. This may also relate to the or a first datum and/or to the or a second datum. The local recalculation can preferentially be carried out randomly, for instance by means of a sampling-rate conversion and/or downsampling.

Consequently the artificial neural network can be trained to deal optimally with data having varying measurement resolution, and to correct said data optimally. This takes into account or enables the application of the method to detected results having varying measurement resolution.

In an advantageous configuration there may be provision that the detected result is corrected, in particular with priority, in the region of a penumbra of a beam profile.

With respect to the correction of the detected result, “with priority in the region of a penumbra” may be understood to mean, in particular, that a difference between the detected result and the corrected detected result is greater in the region of a penumbra than outside this region. This may apply, for instance, to the majority of the data points and/or to a mean value of the data points in the given case.

Consequently the artificial neural network can be trained to correct precisely this region of the beam profile optimally. In this region the detected result or the deviation thereof from an ideal depends especially on the geometry of the detector and its density.

Alternatively or additionally, with a view to achieving the stated object, in accordance with the invention the features of the coordinated claim directed toward a detection arrangement with a detector and with an arithmetic logic unit with an artificial neural network have been provided. In particular, with a view to achieving the stated object in the case of detection arrangements of the type described in the introduction, in accordance with the invention it is consequently proposed that the artificial neural network has been set up to correct a result, detected by means of the detector, of a radiation-physics process pertaining to a radiation-source, and that the artificial neural network has been initially trained with synthetic data pairs, the data pairs comprising a first datum and a second datum, and that the data of a data pair differ by a detector-specific transformation that is uniform for all data pairs.

An equivalent of the initial training of the artificial neural network is contained in this network, for instance in the form of a certain weighting of its artificial neurons.

The first datum may be a synthetic beam profile. The synthetic beam profile may, for instance, describe the ideal of a beam profile. The first datum may furthermore be a beam profile measured with a detector with comparatively high accuracy. The second datum may be a synthetic detection result. The second datum may furthermore be a beam profile measured by means of a detector of comparatively low accuracy, or may describe such a profile. A synthetic data pair contains at least one synthetic datum. For instance, a second datum can be generated synthetically from an actual result of measurement as first datum. This can be effected by application of a detector-specific transformation to the first datum.

By virtue of the fact that the data of a data pair differ by a detector-specific transformation that is uniform for all data pairs, the artificial neural network may have been trained to transform or convert the second datum into the first datum. Consequently the artificial neural network of the detection arrangement can be used to correct a detected beam profile, which is represented by a second datum, for detector-specific influences. By virtue of the fact that the first datum and/or the second datum may be synthetic data, some or all of the previously necessary reference measurements may be dispensable. Consequently there is a saving of time and resources.

In an advantageous configuration there may be provision that the second datum has been generated at least by means of a convolution of the first datum with a lateral response function.

In practice it is the case, as a rule, that the lateral response function with which an ideal can be convolved in order to obtain the actually detected result is known. In addition to convolution, further mathematical operations may be necessary. Whereas the convolution of the first datum—that is to say, of the ideal—with the lateral response function in order to obtain the second datum is comparatively easy to carry out, the deconvolution of the second datum in order to obtain the first datum is not readily possible. The invention has recognized that a plurality of synthetic second data—for instance, detection results—can be easily generated from a plurality of synthetic first data—for instance, beam profiles. These data can then be utilized for training the artificial neural network which can learn to transform the second datum into the first datum. The artificial neural network trained in this way can subsequently be employed in order to correct actually detected results for detector-specific deviations from an ideal.

In an advantageous configuration there may be provision that the first datum has been generated by means of a convolution of a synthetic rough profile with a mapping function.

For instance, the first datum may have been generated by means of a convolution of rectangular profiles and/or rectangular functions with Gaussian functions, preferentially of different width and/or standard deviation.

Consequently, parameters for a synthetic rough profile may have been specified in straightforward manner. The synthetic rough profile may subsequently have been converted into a first datum, for instance a synthetic beam profile. The parameters are, for instance, width parameters, height parameters and/or location parameters of the rough profile.

In an advantageous configuration there may be provision that the lateral response function and/or the mapping function are distribution functions. These may each be, for instance, a Gaussian function. The distribution functions may also be the sum and/or a concatenation of several Gaussian functions. For instance, several convolutions with Gaussian functions may have been carried out.

It has been shown that the detector-specific deviations of a detected result from an ideal can be modeled as a convolution with a Gaussian function. Consequently, first data and second data may have been linked with one another via a Gaussian function. By reason of this relationship, second data such as a synthetic detection result may have been generated from first data such as a synthetic beam profile.

Alternatively or additionally, there may be provision that the synthetic rough profile is a piecemeal linear function. The synthetic rough profile may be, for instance, a rectangular profile and/or a rectangular function. The synthetic rough profile may, for instance, have been chosen to be narrower than a typical leaf width of a multi-leaf collimator. The synthetic rough profile may furthermore have been chosen to be wider than a typical opening cross-section of an aperture. The synthetic rough profile may, for instance, exhibit comparatively few parameters in comparison with the first and second data. The synthetic rough profile may further exhibit supporting points with leaf-width spacing. By means of supporting points with leaf-width spacing, certain values that simulate the position of a leaf of a multi-leaf collimator may have been defined or generated for each supporting point.

In an advantageous configuration there may be provision that the synthetic rough profile has been randomly generated. In this case, there may alternatively or additionally be provision that the mapping function—alternatively or additionally, the first datum—has been randomly generated.

In particular, there may be provision that at least individual (but not necessarily all) parameters of the rough profile and/or of the mapping function and/or of the first datum have been randomly selected, preferentially within a respective specified parameter range. Consequently, not all parameters must necessarily have been chosen randomly in each case. For instance, said parameters may have been partially or completely randomly generated, for instance within certain parameter ranges, or partially defined.

By reason of the convolution of the synthetic rough profile with several Gaussian functions, the parameters of which may have been chosen randomly, various, random forms of the first datum—for instance, of the synthetic beam profile and, in particular, of the penumbral region thereof—may obtain. The randomly varying parameters of the Gaussian functions may be, for instance, the standard deviations thereof or a weighting of the various Gaussian functions.

Consequently a plurality of random first data and second data may be based on a synthetic rough profile exhibiting comparatively few parameters.

In an advantageous configuration there may be provision that a plurality of synthetic rough profiles and/or first data and/or second data, preferably more than 1000 of the respective profiles or data, particularly preferably more than 3000 of the respective profiles or data, have been stored within a single data set for training the artificial neural network.

The profiles or data may consequently have been combined in one data set in resource-saving manner.

In an advantageous configuration there may be provision that the standard deviation of the distribution functions has been restricted to fixed values and/or ranges of values. In particular, this may relate to the Gaussian functions.

Consequently, values that provide realistic results may have been defined. In practice, it may, for instance, be known that for a specific detector an actually detected result has been linked with an ideal by a convolution of the ideal with a Gaussian function having a certain standard deviation.

In an advantageous configuration there may be provision that the or a synthetic rough profile has been furnished with a noise. Alternatively or additionally, there may be provision that the or a first datum and/or the or a second datum has/have been furnished with a noise. The noise has preferentially been randomly generated.

Consequently the synthetic data may also exhibit a noise that, due to the instrument, enters into the detected result in an actual detection arrangement. The artificial neural network may consequently have been trained to correct this noise too. In addition, the diversity of the stated profiles or data can be enhanced further. In this connection, a noise, for instance, may have already been introduced in the case of the synthetic rough profile, or may have been applied to the second datum as end result. For instance, a noise of the synthetic rough profile may have been limited to having been applied only to values of the profile greater than zero, which may correspond to an at least partially opened aperture of a multi-leaf collimator.

In addition, there may be provision that the noise is greater than to be expected in the case of the detected results to be corrected.

Consequently the robustness of the trained artificial neural network and consequently of the method according to the invention can be enhanced.

In an advantageous configuration there may be provision that the or a synthetic rough profile has been recalculated locally at changed resolution. This may also relate to the or a first datum and/or to the or a second datum. The local recalculation may preferentially have been carried out randomly, for instance by means of a sampling-rate conversion and/or downsampling.

Consequently the artificial neural network may have been trained to deal optimally with data having varying measurement resolution, and to correct said data optimally. This takes into account the fact that a detection arrangement according to the invention may comprise detectors having varying measurement resolution.

In an advantageous configuration there may be provision that the corrected detected result has been corrected, in particular with priority, in the region of a penumbra of a beam profile.

With respect to the correction of the detected result, “with priority in the region of a penumbra” may be understood to mean, in particular, that a difference between the detected result and the corrected detected result is greater in the region of a penumbra than outside this region. This may apply, for instance, to the majority of the data points and/or to a mean value of the data points in the given case.

Consequently the artificial neural network may have been trained to correct precisely this region of the beam profile optimally. In this region, the detected result or the deviation thereof from an ideal depends especially on the geometry of the detector and its density.

Alternatively or additionally, with a view to achieving the stated object, in accordance with the invention the features of the coordinated claim directed toward a detection arrangement with means for executing a method as described above have been provided.

Consequently the advantages of the method described previously in the case of detection arrangements can be utilized.

A preferred application of the invention provides that, in one of the methods described above, use is made of a detection arrangement as described above.

Consequently the listed advantages of methods according to the invention and of detection arrangements according to the invention can be combined with one another.

A preferred application of the invention provides that, in the case of methods for measuring radiation with a phantom, a method according to the invention for correcting a result, detected by means of a detector, of a radiation-physics process pertaining to a radiation-source is carried out by an artificial neural network as previously described.

Consequently the previously described advantages of a method according to the invention can be utilized in the case of methods for measuring radiation with a phantom.

Alternatively or additionally, a preferred application of the invention provides that, in the case of methods for measuring radiation with a phantom, use is made of a detection arrangement according to the invention as previously described. Alternatively or additionally, a preferred application of the invention provides that, in the case of methods for measuring radiation with a phantom, a use as previously described is executed.

Consequently the previously described advantages of a detection arrangement according to the invention or of a use according to the invention can be utilized in the case of methods for measuring radiation with a phantom.

Alternatively or additionally, with a view to achieving the stated object, in accordance with the invention the features of the coordinated claim directed toward a phantom have been provided. In particular, with a view to achieving the stated object in the case of phantoms of the type described in the introduction, in accordance with the invention it is consequently proposed that said phantoms exhibit a detection arrangement according to the invention. Alternatively or additionally in this connection, it is proposed that a use according to the invention and/or a method according to the invention for measuring radiation with a phantom is/are executed with the phantoms.

Consequently the previously described advantages of a detection arrangement according to the invention and/or of a use according to the invention and/or of a method according to the invention may have been realized in the case of phantoms of the type described in the introduction.

Alternatively or additionally, with a view to achieving the stated object, in accordance with the invention the features of the coordinated claim directed toward the use of a phantom with a detection arrangement as previously described have been provided. In particular, with a view to achieving the stated object in the case of uses of the type described in the introduction, in accordance with the invention it is consequently proposed that a method as already described is carried out.

Consequently the advantages of a method according to the invention can be utilized in the case where use is made of a phantom.

Alternatively or additionally with respect to the stated object, in accordance with the invention the features of the coordinated claim directed toward an apparatus for generating synthetic data pairs have been provided. In particular, with a view to achieving the stated object in the case of apparatuses of the type described in the introduction, in accordance with the invention it is consequently proposed that synthetic data pairs for a method according to the invention and/or for training a detection arrangement according to the invention, for instance that of a phantom according to the invention, are generated with the apparatus. The training of a detection arrangement or of a phantom is effected with the respective artificial neural networks, which are a constituent part of the respective apparatus.

### BRIEF DESCRIPTION OF THE DRAWINGS

The invention will now be described in more detail with reference to embodiments, but it is not limited to the embodiments. Further embodiments arise through combination of the features of individual or of several claims with one another and/or with individual or several features of the embodiments.

FIG. 1 shows a detection arrangement according to the invention,

FIG. 2 shows a further detection arrangement according to the invention,

FIG. 3 shows a flowchart of a training and of a functional operation of the artificial neural network, and

FIG. 4 shows a flowchart of an operational application of the trained artificial neural network.

### DETAILED DESCRIPTION

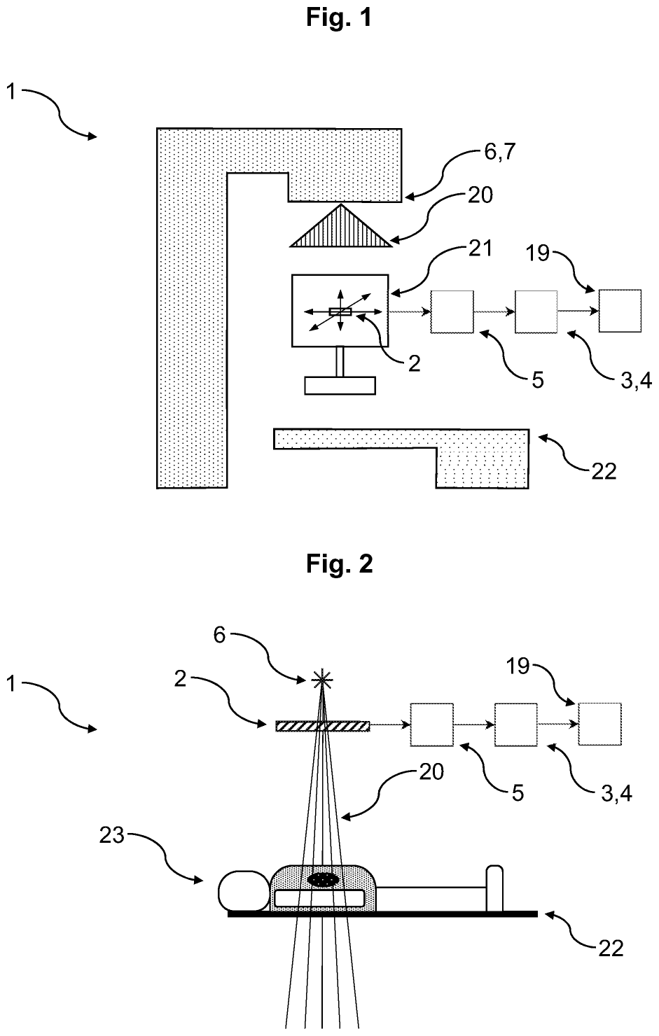

The detection arrangement 1 according to the invention represented in FIG. 1 exhibits means for executing a method according to the invention and can consequently be used in such a method. The detection arrangement 1 comprises a detector 2 and an arithmetic logic unit 3 with an artificial neural network 4. The artificial neural network 4 has been set up to correct a result 5, detected by means of the detector 2, of a radiation-physics process pertaining to a radiation-source 6, whereby a corrected detected result 19 is obtained. The radiation-source 6 in this case is a linear accelerator 7 which emits a particle beam 20. The radiation-source 6 may comprise, for instance, an electron-source and/or ion-source, not shown in any detail.

FIG. 1 further shows a phantom 21 according to the invention, namely with a detection arrangement 1. The phantom 21 is suitable for a use, according to the invention, of a detection arrangement 1 in a method according to the invention. For instance, a method according to the invention for measuring radiation can be executed with the phantom 21, wherein a method according to the invention is executed and/or a detection arrangement 1 according to the invention is used and/or a use according to the invention is executed. A phantom 21 is conventionally employed in order to carry out monitoring measurements while a patient table 22 is available to support a patient 23 during a radiation treatment.

FIG. 2 shows a further detection arrangement according to the invention with a radiation-source 6, with a detector 2, with a particle beam 20 and with a patient table 22 with a patient 23. Also in this embodiment, a result 5, detected by means of the detector 2, of a radiation-physics process pertaining to a radiation-source 6 is passed for correction to an arithmetic logic unit 3 with an artificial neural network 4, after which a corrected detected result 19 is obtained.

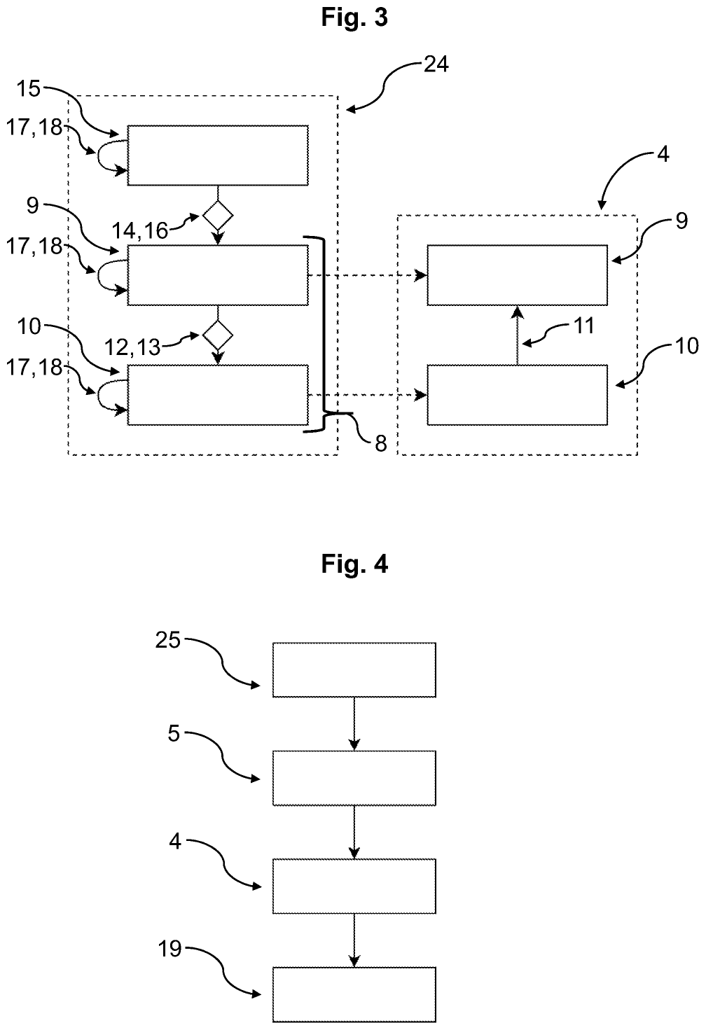

The artificial neural network 4 has been or is initially trained with synthetic data pairs 8, the data pairs 8 comprising a first datum 9 and a second datum 10, and the data 9, 10 of a data pair 8 differing by a detector-specific transformation 11 that is uniform for all data pairs 8. This is evident in the flowchart in FIG. 3. FIG. 3 also reproduces the sequence of operations of a method according to the invention for correcting a result 5, detected by means of a detector 2, of a radiation-physics process pertaining to a radiation-source 6 by an artificial neural network 4. The objective of the training of the artificial neural network 4 is consequently the ascertainment of the uniform detector-specific transformation 11. The latter is contained in the trained artificial neural network 4, for instance in the form of a weighting of its artificial neurons. An artificial neural network 4 trained in such a manner can subsequently be used for correcting a detected result 5 in order to obtain a corrected detected result 19.

The synthetic data pairs 8 may, for instance, have been or be generated with an apparatus 24 according to the invention for generating synthetic data pairs.

The second datum 10 has been or is generated at least by means of a convolution 12 of the first datum 9 with a lateral response function 13. The first datum 9 has been or is generated by means of a convolution 14 of a synthetic rough profile 15 with a mapping function 16.

The lateral response function 13 and/or the mapping function 16 may be distribution functions, for instance a Gaussian function or the sum and/or concatenation of several Gaussian functions. The synthetic rough profile 15 may be a piecemeal linear function.

The synthetic rough profile 15 and/or the mapping function 16 and/or the first datum 9 may have been or may be randomly generated.

An advantageous manipulation of the synthetic rough profiles 15, first data 9 and of the second data 10 may result if a plurality, for instance more than 1000 or more than 3000, of the respective profiles 15 or data 9, 10 have each been stored within a single data set for training the artificial neural network 4.

The standard deviations of the distribution functions, in particular of the Gaussian functions, may have been restricted to fixed values and/or ranges of values.

Furthermore, the synthetic rough profile 15 and/or the first datum 9 and/or the second datum 10 may have been or may be furnished with a noise. The furnishing with a noise 17 may be part of a method according to the invention. The noise may, for instance, have been or be randomly generated and/or may be greater than to be expected in the case of the detected results 5 to be corrected.

Furthermore, the synthetic rough profile 15 and/or the first datum 9 and/or the second datum 10 may have been or may be recalculated locally at changed resolution. This recalculation 18 may have been or may be effected randomly, for instance by means of a sampling-rate conversion and/or downsampling.

The corrected detected result 19 may, for instance, have been or be corrected in the region of a penumbra of a beam profile.

FIG. 4 shows a flowchart of an application of a detection arrangement according to the invention or of a phantom according to the invention or of a method according to the invention. After a, for instance, manually triggered start 25 of a measurement, a detected result 5 is obtained which is transferred to the artificial neural network 4 which executes the uniform detector-specific transformation 11 and in this way provides a corrected detected result 19.

Consequently a method is proposed for correcting a result 5, detected by means of a detector 2, of a radiation-physics process pertaining to a radiation-source 6 by an artificial neural network 4, wherein the artificial neural network 4 was initially trained with synthetic data pairs 8, wherein the data pairs 8 comprise a first datum 9 and a second datum 10, and wherein the data 9, 10 of a data pair 8 differ by a detector-specific transformation 11 that is uniform for all data pairs 8. Furthermore, a detection arrangement 1 with a detector 2 and with an arithmetic logic unit 3 with an artificial neural network 4 is proposed, wherein the artificial neural network 4 has been set up to correct a result 5, detected by means of the detector 2, of a radiation-physics process pertaining to a radiation-source 6, and wherein the artificial neural network 4 has been initially trained with synthetic data pairs 8, wherein the data pairs 8 comprise a first datum 9 and a second datum 10, and wherein the data 9, 10 of a data pair 8 differ by a detector-specific transformation 11 that is uniform for all data pairs 8. Furthermore, the use is proposed of a detection arrangement 1 according to the invention in a method according to the invention. Moreover, a method for measuring radiation with a phantom 21, and a phantom 21 with a detection arrangement 1, are proposed, wherein in each case methods and/or uses according to the invention are executed and/or apparatuses according to the invention have been constructed. Furthermore, an apparatus is proposed for generating synthetic data pairs 8 for a method according to the invention and/or for training a detection arrangement 1 according to the invention and/or for training a phantom 21 according to the invention.

### LIST OF REFERENCE SYMBOLS

1 detection arrangement
2 detector
3 arithmetic logic unit
4 artificial neural network
5 detected result
6 radiation-source
7 linear accelerator
8 synthetic data pair
9 first datum
10 second datum
11 detector-specific transformation
12 convolution
13 lateral response function
14 convolution
15 synthetic rough profile
16 mapping function
17 furnishing with a noise
18 recalculation
19 corrected detected result
20 particle beam
21 phantom
22 patient table
23 patient
24 apparatus for generating synthetic data pairs
25 start

1. A method for correcting a result (5) of a radiation-physics process pertaining to a radiation-source (6) by an artificial neural network (4), the method comprising:
detecting a result using a detector (2);
initially training the artificial neural network (4) with synthetic data pairs (8), wherein the synthetic data pairs (8) comprise a first datum (9) and a second datum (10); and
the data (9, 10) of one said data pair (8) differ by a detector-specific transformation (11) that is uniform for all said data pairs (8).

2. The method as claimed in claim 1, further comprising generating the second datum at least using a convolution (12) of the first datum (9) with a lateral response function (13).

3. The method as claimed claim 2, further comprising generating the first datum (9) using a convolution (14) of a synthetic rough profile (15) with a mapping function (16).

4. The method as claimed in claim 3, wherein at least one of a) at least one of the lateral response function (13) or the mapping function (16) are distribution functions, or b) the synthetic rough profile (15) is a piecemeal linear function.

5. The method as claimed in claim 4, wherein at least one of the synthetic rough profile (15), the mapping function (16), or the first datum (9) is randomly generated, with at least individual parameters of the at least one of the rough profile (15), the mapping function (16), or the first datum (9) being randomly selected.

6. The method as claimed in claim 4, further comprising using a plurality of at least one of a) said synthetic rough profiles (15), b) said first data (9), or said second data (10) within a single data set for training the artificial neural network (4).

7. The method as claimed in claim 4, wherein the at least one of the lateral response function (13) or the mapping function (16) are distribution functions, and standard deviations of the distribution functions that are restricted to at least one of fixed values or ranges of values.

8. The method as claimed in claim 3, further comprising furnishing at least one of the synthetic rough profile (15), the first datum (9), or the second datum (10) with randomly generated noise that is greater than an expected noise for the detected results (5) to be corrected.

9. The method as claimed in claim 3, further comprising recalculating at least one of the synthetic rough profile (15), the first datum (9), or the second datum (10) locally at a changed resolution by at least one of a sampling-rate conversion or downsampling.

10. The method as claimed in claim 9, further comprising correcting the detected result (5) in a region of a penumbra of a beam profile.

11. A detection arrangement (1), comprising:
a detector (2);
an arithmetic logic unit (3) with an artificial neural network (4), the artificial neural network (4) being configured to correct a result (5), detected using the detector (2), of a radiation-physics process pertaining to a radiation-source (6);
wherein the artificial neural network (4) is initially trained with synthetic data pairs (8), the data pairs (8) comprising a first datum (9) and a second datum (10), and the data (9, 10) of one said data pair (8) differ by a detector-specific transformation (11) that is uniform for all said data pairs (8).

12. The detection arrangement (1) as claimed in claim 11, wherein the arrangement is configured such that the second datum (10) has been generated at least using a convolution (12) of the first datum (9) with a lateral response function (13).

13. The detection arrangement (1) as claimed in claim 12, wherein the arrangement is configured such that the first datum (9) has been generated using a convolution (14) of a synthetic rough profile (15) with a mapping function (16).

14. The detection arrangement (1) as claimed in claim 13, wherein the arrangement is configured such that at least one of a) at least one of the lateral response function (13) or the mapping function (16) are distribution functions, or b) the synthetic rough profile (15) is a piecemeal linear function.

15. The detection arrangement (1) as claimed in claim 14, wherein the arrangement is configured such that at least one of the synthetic rough profile (15), the mapping function (16), or the first datum (9) was randomly generated, and at least individual parameters of at least one of the rough profile (15), the mapping function (16), or of the first datum (9) were randomly selected within a respective specified parameter range.

16. A detection arrangement (1) as claimed in claim 14, wherein the arrangement is configured with at least one of a plurality of synthetic rough profiles (15), said first data (9), or second data (10) stored within a single data set for training the artificial neural network (4).

17. The detection arrangement (1) as claimed in claim 14, wherein the arrangement is configured such that standard deviations of the distribution functions have been restricted to at least one of fixed values or ranges of values.

18. The detection arrangement (1) as claimed in claim 14, wherein the arrangement is configured such that at least one of the synthetic rough profile (15), the first datum (9), of the second datum (10) has been furnished with randomly generated noise that is greater than to be expected for the detected results (5) to be corrected.

19. The detection arrangement (1) as claimed in claim 14, wherein the arrangement is configured such that at least one of the synthetic rough profile (15), the first datum (9), of the second datum (10) has been recalculated locally at a changed resolution by at least one of a sampling-rate conversion or downsampling.

20. The detection arrangement (1) as claimed in claim 14, wherein the arrangement is configured such that a corrected detected result (19) is corrected in a region of a penumbra of a beam profile.

21. A method for measuring radiation with a phantom (21), the method comprising carrying out the method as claimed in claim 1.

22. A phantom (21) comprising the detection arrangement (1) as claimed in claim 11.
